(19) United States
(12) Patent Application Publication
(10) Pub. No.: US 2020/0197282 A1
(43) Pub. Date: Jun. 25, 2020

(54) BATH SALT COMPOSITION AND METHOD OF MAKING THEREOF

(71) Applicant: Charlene Ashmore, Atwater, CA (US)

(72) Inventor: Charlene Ashmore, Atwater, CA (US)

(21) Appl. No.: 16/224,857

(22) Filed: Dec. 19, 2018

(51) Int. Cl. A61K 8/49; A61P 29/00; A61K 31/352

(52) U.S. Cl. CPC A61K 8/498; A61Q 19/10; A61K 31/352; A61P 29/00

(57) ABSTRACT

A bath salt composition and method of making thereof includes providing dried Cannabis leaves containing THC and defining THC grind and dried Cannabis leaves having deactivated THC and defining THCA grind. The THC grind and the THCA grind are ground together to define a mixed grind. The mixed grind is blended with 8 parts refined avocado oil and cooked define a cooked mixture. The THC grind and THCA grind are strained from the cooked mixture to define a strained mixture which is poured onto Epson salt to define a composition. The composition is dried and placed into an airtight container until used in bathwater.

### CROSS-REFERENCE TO RELATED APPLICATIONS

Not Applicable

### STATEMENT REGARDING FEDERALLY SPONSORED RESEARCH OR DEVELOPMENT Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention.

### (2) Description of Related Art Including Information Disclosed Under 37 CFR 1.97 and 1.98.

The disclosure and prior art relates to pain relief bath salt compositions and more particularly pertains to a new pain relief bath salt composition for aiding in the reduction of pain experienced by a person's body.

### BRIEF SUMMARY OF THE INVENTION

An embodiment of the disclosure meets the needs presented above by generally providing dried Cannabis leaves containing THC and defining THC grind and dried Cannabis leaves having deactivated THC and defining THCA grind. The THC grind and the THCA grind are ground together to define a mixed grind. The mixed grind is blended with 8 parts refined avocado oil and cooked define a cooked mixture. The THC grind and THCA grind are strained from the cooked mixture to define a strained mixture which is poured onto Epson salt to define a composition. The composition is dried and placed into an airtight container until used in bathwater.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

Not Applicable

### DETAILED DESCRIPTION OF THE INVENTION

With reference now to the description below, a new pain relief bath salt composition embodying the principles and concepts of an embodiment of the disclosure will be described.

The bath salt composition and method of making thereof generally comprises the steps of grinding and mixing together two different Cannabis ingredients, or leaves. One of the Cannabis ingredients contains active THC (tetrahydrocannabinol) and may more particularly be a strain which is known by the name CBD-OC typically having a THC content level of between 15% and 17%. For purposes of clarity, this ingredient will be defined as THC grind. While other strains of Cannabis leaves may be used, the THC content should typically be at least 15%.

Another of Cannabis ingredients being used contains little to no THC and instead comprises THCA (tetrahydrocannabinolic acid) which does not have the psychoactive properties of THC. For clarity, this ingredient will be defined as THCA grind and will typically have a THCA content of at least 15% and will more generally have a THCA content of between 15% and 21% and may be derived from the Cannabis indica strain whereas the THC grind may be derived from the Cannabis sativa strain.

Generally, 1 part by weight of THC grind is mixed with 1 part by weight THCA grind and ground together to define a mixed grind. This mixed grind is then mixed with avocado oil and more specifically may be mixed with refined avocado oil. Avocado oil is readily available and is made from the pressed fruit of the avocado tree (Persea americana). The ratio by weight is typically 1 part THC grind, 1 part THCA grind and 8 parts refined avocado oil.

This mixture of THC, THCA and avocado oil is then heated at a temperature of between 60° C. and 83° C. The time for heating may vary as it will last until the THC and THCA grinds have hardened. Typically this time frame is at least 45 minutes and less than 75 minutes. Once removed from the heat, a cooked mixture is defined and is allowed to cool to room temperature of approximately 16° C. to 25° C. The cooked mixture is strained in a conventional manner such as through a mesh net, cheesecloth or an extremely fine strainer to remove any remaining ground leaves. The remaining fluid, after the THC grind and THCA grind have been removed, is defined as a strained mixture.

The strained mixture will then be poured onto Epson salt (magnesium sulfate or epsomite MgSO₄7H₂0), and allowed to dry. To aid in this process, the Epson salt may be spread out on multiple pans or other flat surfaces and the strained mixture poured onto the Epson salt. The quantity of Epson salt is typically 120 to 200 parts Epson salt to the 1 part THC grind though 160 parts of Epson salt will typically be the amount used.

After the strained mixture is poured over onto the Epson salt, a composition is defined that is allowed to dry. The drying process can take between 24 hours and 120 hours, though this time is dependent upon ambient temperature and humidity and therefore may be hastened with a dry heat applied to the composition. Once dried, the composition is placed in airtight containers until used.

As a more specific example of the above the method may include the steps of:
1) Providing a double boiler having an inner pot and an outer pot;
2) Placing 2 quarts of water in the outer pot and heating such to between 60° C. and 83° C.;
3) Placing in the outer pot 1.0 ounces of the THC grind, 1.0 ounces THCA grind to further define the mixed grind, and adding 8.0 ounces of refined avocado oil to the mixed grind;
4) Placing the outer pot into the inner pot and heating the inner pot for 1.0 hours;
5) Removing the outer pot from the inner pot and allowing the outer pot and the cooked mixture therein to cool to room temperature;
6) Straining the cooked mixture to remove the THC grind and THCA grind to define the strained mixture;

7) Pour the strained mixture onto 10.0 pounds of Epson salt to define the composition; and
8) Air drying the composition for between 3.0 and 5.0 days, storing such in an airtight container(s) and using as needed thereafter.

In use, a person will add the composition to their bathwater when taking a bath. The amount added will typically be between 1 and 3 teaspoons (approximately 5 to 15 ml). The bathwater is then used in a conventional fashion while the composition aids in reducing pain in the person's body, particularly associated with skeletal and muscular pain.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A method for making a bath salt for placing in a bathtub to relieve a person's pain, said method comprising the steps of:
providing one part by weight dried Cannabis leaves containing THC and defining THC grind;
providing one part by weight dried Cannabis leaves having deactivated THC and defining THCA grind;
grinding together said THC grind and said THCA grind to define a mixed grind;
mixing said mixed grind with 8 parts refined avocado oil;
heating said mixed grind and said refined avocado oil to define a cooked mixture;
straining the THC grind and THCA grind from the cooked mixture to define a strained mixture;
pouring said strained mixture Epsom salt to define a composition;
drying said composition; and
storing said composition in airtight containers.

2. A method for making a bath salt for placing in a bathtub to relieve a person's pain, said method comprising the steps of:
providing by weight 1 part dried Cannabis leaves containing THC and defining THC grind;
providing by weight 1 part of dried Cannabis leaves having deactivated THC and defining THCA grind;
grinding together said THC grind and said THCA grind to define a mixed grind;
mixing said mixed grind with 8 parts by weight refined avocado oil;
heating said mixed grind and said refined avocado oil at a temperature of between 60° C. and 83° C. for a time frame of between 45 minutes and 75 minutes to define a cooked mixture;
straining the THC grind and THCA grind from the cooked mixture to define a strained mixture;
pouring said strained mixture onto between 120 parts and 200 parts by weigh of Epsom salt to define a composition;
drying said composition between 24 hours and 120 hours; and
storing said composition in airtight containers.
